(19) United States
(12) Patent Application Publication
(10) Pub. No.: US 2008/0009732 A1
(43) Pub. Date: Jan. 10, 2008

(54) Process of using a direct imaging apparatus (like ultrasound catheter or fiber-optic/hysteroscopic imaging) for real time intra-vaginal imaging for intra-partum assessment of cerrvical dilatation and descent of fetal presenting part and any other management of active labor with the goal of delivery

(76) Inventor: Bolanle Adekore SOGADE, Macon, GA (US)

Correspondence Address: Dr.BOLANLE ADEKORE SOGADE, SUITE 101, 639 HEMLOCK STREET, MACON, GA, 31201, US

(21) Appl. No.: 11/306,589

(22) Filed: Jan. 3, 2006

(51) Int. Cl. A61B 8/00

(52) U.S. Cl. 600/443

(57) ABSTRACT

Any imaging apparatus like a real-time three dimensional ultrasound imaging catheter apparatus or any small lighted fiber-optic instrument; or the like is: positioned to view the cervical os and the presenting part of the fetus and the placed in any area in or around the female pelvis or in any related cavities i.e the urinary bladder; or in itself designed as e.g a urinary catheter, comprising a proximal and distal end. The distal end is connected through a small elongated body to a proximal end. The proximal end is connected to any apparatus or means that is calibrated to record,measure and interpret the data of cervical dilatation and effacement; and the descent of he fetal presenting part; including but not limited to a monitor and printer; or eye piece/camera

### FIELD OF THE INVENTION

The present invention relates to the field of imaging in general and to catheter ultrasound imaging and direct endoscopic imaging in particular with respect to measuring the dilatation of the human cervix and the descent of the presenting part of the fetus during labor/parturition and other aspects of labor management.

### BACKGROUND OF THE INVENTION

Assessment of the progress of labor has traditionally meant repeated and serial digital pelvic examinations. The knowledge of the state of the cervix (the effacement and dilatation) and the station of the presenting part of the fetus is critical to know because normal as well as abnormal labor that will require surgical intervention can be diagnosed.

These serial digital examinations have been performed by varied labor and delivery personnel (nurses midwives obstetricians, family physicians and medial students) with varied levels of experience; at great discomfort to the patient and with great inter-observer variations (even when performed by obstetricians with the same level of experience).

The resultant subjective measurements have varied; and were not reproducible, even immediately. There are risks to repeated digital examination including the introduction of infection of the fetal membranes (chorioamnionitis) and subsequently the whole fetus, (neonatal sepsis) especially in preterm infants in which the plan is to prolong the duration of the gestation to fetal maturity; and infection of the lining and/or muscle of the uterus (endomyometritis).

Previous inventions U.S. Pat. No. 6,039,701 have attempted to use mechanical means to attach materials to the cervix with the attendant problems of adherence to the cervix and sheer patient discomfort It is therefore desirable to provide a simple means of measuring the cervix at the least patient discomfort Due to the short propagation distance, high frequency imaging and accurate tissue characterization using high frequency (e.g. 7-10 MHz) imaging is possible in trans-vaginal scans. Also, ultrasonic needle guidance may be accomplished trans-vaginally for the total management of labor. This will including for artificial rupture of the membranes to augment the pace of labor, systematic replacement of meconium-stained amniotic fluid by intra-amniotic transfusion of normal saline through an integrated delivery system, and any other procedure in the comprehensive management of labor; all performed in a manner that is more safely and less painfully.

Additionally, since less intervening tissue is between the probe and target the signal-to-noise ratio is greatly improved, especially in obese patients.

One intracavity probe now commercially available employs a mechanical section scanning element mounted inside an elongated housing for rotation therein. The scanning head is relatively large and the image quality is marginal. The probe does accommodate a biopsy needle. Another known probe includes an oscillating transducer in an elongated housing. To alter the image scan direction the elongated housing must be axially tilted in the vaginal cavity, causing discomfort and pain to the patient. The probe does not function as a biopsy needle guide. Ultrasonic imaging has been applied in many two dimensional systems using pulse echo B-mode tomography or B-scans. These systems display echoes returning to an ultrasonic transducer as brightness levels proportional to echo amplitude. The brightness levels may be used to create cross-sectional images of the object in the plane perpendicular to the transducer aperture.

Examination of objects in three dimensions has evolved using a number of modalities including xray, ultrasound, and nuclear magnetic resonance. In particular, improvements have been made in spatial resolution, dynamic range, display methods and data analysis. For example, ultrasound scanning of three-dimensional objects by sequential B-scans followed by off-line reconstruction and display of rendered images has progressed in recent years with the introduction of commercial three-dimensional systems. Off-line rendering, however, may take several minutes to produce a single three-dimensional scan.

In the area of high-speed three-dimensional ultrasound imaging, U.S. Pat. No. 4,596,145 to Smith and von Ramm discloses an acoustic imaging system capable of producing high-speed projection orthoscopic images, as well as a single high-speed C-scan image using a two-dimensional array transducer and receive mode parallel processing. The C-scan image may be defined as a planar section of the object parallel to the effective transducer aperture. In 1987, U.S. Pat. No. 4,694,434 to von Ramm and Smith disclosed a steered array acoustic imaging scanner capable of producing a high-speed pyramidal scan to obtain a volumetric (three-dimensional) image using a two-dimensional array transducer and receive mode parallel processing. High frequency intraluminal ultrasound imaging probes have been developed, including circular arrays and mechanically steered transducers. The circular arrays and mechanically steered transducers produce B-mode circular side scan geometries in which the ultrasound beam is swept through a 360.degree. arc. The 360.degree. arc may create a high-speed circular image within a vessel or lumen with a maximum range of approximately one centimeter. For example, U.S. Pat. No. 3,938,502 to Bom and U.S. Pat. No. 4,917,097 to Proudian, et al. disclose circular arrays of transducer elements within a catheter to produce a circular side scanning intraluminal Bmode image. U.S. Pat. No. 4,794,931 to Yock and U.S. Pat. No. 5,243,988 to Sieben, et al. disclose motor-driven piston transducers at the end of the catheters to produce circular side scanning intervascular imaging.

Catheters may be used in conjunction with the systems described above to provide intraluminal imaging. Intraluminal imaging may involve inserting a catheter, that includes an ultrasonic transducer phased array, into coronary vessels, pulmonary arteries, the aorta, or venous structures. For example, U.S. Pat. No. 5,704,361 to Seward, et al. discloses a volumetric imaging ultrasound transducer under-fluid catheter system. The advantages of Seward may, however, be limited by the quality of the imaging provided therein. In particular, the catheter probes disclosed in Seward show the therapeutic tools adjacent to the transducer array on the catheter tip, thereby reducing the area available for the transducer array. Such an array may provide images having reduced spatial resolution. Moreover, the applications described in Seward may be limited to procedures involving catheters. The catheters described above may be combined with electrodes or tools to locate (the position of the fetal presenting part in relation to e.g. the ischial spines) and perform therapy on (rupture a bulging bag of membranes) or monitor tissue (the dilatation of the cervix). For example, a three-dimensional ultrasound imaging device using a catheter may be combined with an electrode to provide therapy to particular tissue. The therapy provided by the electrode, however, may be limited by the registration between the image provided by the catheter and the electrodes associated with the catheter. For example, a user may have difficulty translating the image produced by the catheter to the position of the electrode, thereby possibly creating difficulty in applying the electrode to the intended tissue. Moreover, the electrode may obscure the three dimensional ultrasound image when the electrode is within the field of view of the image.

Also, now in the era of ultra-slim and flexible fiber-optic imaging in the form of the hysteroscope/endoscope, a very small and flexible imaging catheter like a fiber-optic hysteroscope can be placed with minimal discomfort even into a nulliparous vagina and used to provide direct imaging of the changes in the cervix and other management of labor in real time.

There will be less deliveries ‘missed’ because the obstetrician was not notified on time or because the cervix changed at a quicker than anticipated rate of change

In view of the above discussion, there exists a need to measure the progress of labor as assessed by measuring the dilation of the cervix, in a reproducible and standardized, quantifiable means and the descent of the fetal presenting part (usually the head or rarely the breech of the fetus) in a manner that precludes repetitive digital cervical examinations and the attendant extreme discomfort to the laboring patient with improved quality real-time three-dimensional imaging in an intra-vaginal or intra-urinary bladder catheter ultrasound applications; or by direct imaging in any form including but not limited to fiber-optic direct visualization of the lower uterine segment, the cervix and direct interventions in all the aspects of management of labor/parturition

What is claimed is:

1. A) Any imaging apparatus like but not limited to:
1) a real-time three dimensional ultrasound imaging catheter apparatus or
2) a small hysteroscope or any lighted fiber-optic instrument; or the like, of small enough caliber to placed in the nulliparous vagina without discomfort; and is
B) positioned to view the vagina specifically the lower uterine segment including the cervical os and the presenting part of the fetus and
C) the placement can be in anywhere therein listed but not limited to:
1) in the vaginal cavity; or
2) via a Foley catheter into the urinary bladder or in itself designed as a Foley catheter, or
3) into the ‘prepared’ rectum, or 4) just on the perinuem
The apparatus comprises a flexible, elongated body, like but not limited to a catheter, or a semi-rigid or flexible hysteroscope having proximal and distal ends.

2. The distal end is connected to the elongated body and consists of, but is not limited to:
1) an ultrasonic transducer phased array; or
2) an objective lens or digital camera assembly similar to, but not limited to an hysteroscope/endoscope; or
3) any apparatus or substance that will:
A) image or give information on the status (for example dilatation and effacement) of the cervical os and the presenting fetal part (for example descent); and
B) can also be used to manage any aspect of labor.

3. Examples are:
1) administering fluids or medications to the uterus, amniotic fluid, the fetus or the mother; rupturing the fetal membranes;
2) recording any data from the fetus like fetal heart rate or uterine contraction patterns, oxygenation levels,
3) to assess substances in the amniotic fluid or in the fetal blood like meconium or to assess for infection of the fluid and the likes
The ultrasonic transducer phased array (of transducer elements) is positioned to emit and receive ultrasonic energy for volumetric forward scanning from the distal end of the elongated body, whereby the imaging field of view is provided by rotating the catheter/probe; or using 0-degree for distant panoramic view or angled—any angle for a better view.

4. The ultrasonic transducer phased array includes a plurality of sites occupied by ultrasonic transducer elements. At least one ultrasonic transducer element is absent from at least one of the sites, thereby defining an interstitial site.

5. A tool is positioned at the interstitial site for measurements like of fetal heart rates, artificial rupture of membranes administration of fluids or medications; and the sort. In particular, the tool can be a fiber optic lead, a suction tool, a guide wire, an electrode of any type.

6. The proximal end is connected to any apparatus or means that can record and interpret the data of cervical dilatation, effacement or the descent of he fetal presenting part; including but not limited to monitor and printer; or eye piece/camera.
